(19) United States
(12) Patent Application Publication
(10) Pub. No.: US 2024/0100273 A1
(43) Pub. Date: Mar. 28, 2024

(54) DEVICE FOR TRANSFERRING ENERGY BETWEEN EXHALING AND INHALING PHASES

(71) Applicant: SRS SERVIÇOS RELACIONADOS A SAUDE LTDA, Mogi das Cruzes (BR)

(72) Inventor: Pedro Yoshitaka Fukuyama, Mogi das Cruzes (BR)

(21) Appl. No.: 18/276,147

(22) PCT Filed: Feb. 8, 2022
(86) PCT No.: PCT/BR2022/050038

§ 371 Date: Aug. 7, 2023

(30) Foreign Application Priority Data
Feb. 10, 2021 (BR) 102021002545-0

(51) Int. Cl. A61M 16/00; A61M 15/00; A61M 16/20

(52) U.S. Cl. CPC A61M 16/0003; A61M 15/0086; A61M 16/0057; A61M 16/201; A61M 2016/0015; A61M 2205/076

(57) ABSTRACT

A device that reduces human energy loss in the breathing process, without requiring external energy such as electricity. The device transfers motion, elastic or heat energy by conveying air volume and pressure between the exhaling and inhaling phases in the breathing process in order to adjust the air pressure or temperature in these phases so as to improve air perfusion or the body's thermal homeostatic conditions as required, in addition to reducing the air resistance in masks or respirators used for protection against contaminants. The invention pertains to the field of healthcare. The invention comprises inhalation and exhalation chambers that are associated with air pumps, besides having elastic energy accumulation walls to minimize air flow resistance.

The objective of the present application is to propose a device that reduces human energy loss in the breathing process without requiring external energy such the electricity or outside air pressure. The product conceived is a breathing mask that enables positive pressure in exhaling and in inhaling or that enables a reduction in air resistance in breathing mask filters during the air inhaling or exhaling phase. The device transfers pneumatic, motive or elastic energy by conveying air volume and pressure between the exhaling and inhaling phases in the breathing process in order to adjust the air pressure in these phases so as to improve air perfusion and the body's homeostatic conditions as required. One of its consequences is to reduce air resistance in respirators or respiratory protection masks against biological, physical or chemical agents or to improve the breathing process in respiratory pathologies. It pertains to the field of healthcare and is useful in situations of endemics such as that of Covid-19 and also in normal situations for treating respiratory pathologies. In its preferred embodiments, it enables positive pressure in exhaling and in inhaling and lesser air resistance in breathing mask filters.

Disease situations refer to pulmonary diseases in general, chiefly those that alter the resistive forces of the airways and also the elastic forces (such as those that influence pulmonary complacency and movement of the rib cage). Sleep apnea, asthma, bronchitis, pulmonary emphysema and pulmonary fibrosis are examples of pathologies that act on these forces. It also occurs in other pathologies such as obesity. Hypothermia situations also adversely affect the metabolism of living beings.

Adverse environmental situations are those in which environments occur with prejudicial chemical, physical or biological elements. In these situations, air filters are required to retain these contaminants, increasing the air resistance which may cause respiratory fatigue. In times of Covid-19, what most draws attention are biological elements such as viruses, bacteria and pathological fungi. The smaller the porosity of the filters, the more effective the filtering, yet it increases the air flow resistance a lot, meaning its use is limited. Oftentimes, patients with respiratory pathologies can be hospitalized in contaminated environments such as common or infectology wards, or intensive care units. So the use of air filters may be necessary, which increases the air flow resistance, this being a worsening factor for patients with respiratory pathologies.

Masks with air filters protect whoever uses them, but also protects those around them insofar as they prevent a contaminated patient from contaminating others. In this case, by using masks in exhaling, an increase in air resistance also occurs, which may trigger discomfort or respiratory fatigue.

The dynamics of breathing are highly complex, and the need often arises for artificial interventions performed by respirators, which increase or reduce the pressure in the inhaling or exhaling phase. Each pathology or adverse outside environment has different needs. For example, one beneficial intervention is the artificial introduction of positive pressure in exhaling, a measure that may reduce alveolar collapse and expand the airways in exhaling. Positive pressure on inhaling facilitates the entry of air, chiefly in obstructive airway pathologies or in airway collapse pathologies producing snoring and sleep apnea. In other situations, it is useful to have positive or negative pressure. The best balance between air pressures, between inhaling and exhaling, reduces respiratory fatigue and saves lives.

Relative to the state of the art, the following solutions are set forth.

Artificial respirators are known to use air pumps producing positive or negative pressure, having pressure sensors, flow and volume, powered by external energy, most often by electricity. But they are complex, expensive and use a lot of external energy.

Expiratory Positive Airway Pressure (EPAP) devices are known to be capable of producing positive pressure in the exhaling phase, and generally use an air valve to generate air resistance in exhaling. Continuous Positive Airway Pressure (CPAP) devices are capable of producing artificial pressures in inhaling and exhaling phases, and generally use electric air pumps or ventilators powered by electricity. Bilevel Positive Airway Pressure (BIPAP) devices provide pressure levels adjustable to different phases, exhaling or inhaling, generally using pressure sensors to verify the pressure levels and electric air pumps.

The traditional artificial respirators CPAP and BIPAP are heavy (even the portable ones), consume external energy, are complex and expensive. EPAP devices are simpler, but in general do not help with inhaling.

For adverse external environment situations, devices with air filters such as traditional respirators and face masks serve to filter the air, filtering contaminants. They bear the drawback of increasing air resistance.

One of the strategies already known in the state of the art is the increase of the air filter change surface, however this solution is limited because this increase cannot increase indefinitely due to limitations of space, weight and cost. This filtering material is generally expensive.

Bearing in mind these drawbacks and with a view to solving them, this application devises solutions to solve or mitigate these issues.

This application consists of using two solutions which together make up a unit capable of minimizing the breathing effort as far as possible, preferably without the action of external energy such as electricity or that coming from gas reservoir pressure, in addition to being very low cost. These solutions can also be used separately. A shared solution has been devised that refers to air distribution chambers in inhaling and in exhaling, having a shared objective which is the transfer of (pneumatic) energy between these phases (of inhaling and exhaling), and these chambers are the basis of defense of inventive unity of this application.

For the shared solution, a device has been conceived, comprising a face mask which is coupled to the face and connects to a chamber for exhaling, having a valve that enables the one-way passage of air towards the face mask to the inside of the chamber. It also has a chamber for inhaling, having a valve that enables the one-way passage of air leaving the inside of the chamber towards the face mask. They will be better illustrated in the drawings and were conceived to enable the volumes of exhaling and inhaling air to be separated and directed to the following solutions.

The first of these solutions conceived the coupling of inhaling and exhaling chambers to mechanical pneumatic pumps. A pump receiving exhaling chamber air passes mechanical energy to another pump injecting air into inhaling chambers. Accordingly, in the preferred arrangement of this application, a pressure increase is promoted in exhaling, reducing the collapse of the alveoli and air channels (windpipe, for example), while simultaneously increasing the air pressure in the inhaling phase, facilitating the capture of air by the lungs.

This first solution was devised by looking for extra resources in nature. It was noted that the muscles that enable exhaling have an additional energy reserve to be used to help inhaling. These exhaling muscles have a greater reserve because they have additional resources so that humans can speak, shout and sing, so they have a robust musculature that exceeds their mere exhaling respiratory function. This solution sought to utilize this muscular potential reserve to generate pneumatic energy (positive pressure) to assist in the inhaling phase without tiring the exhaling musculature because the latter has this additional reserve.

In the second solution, it was determined that there are different and exclusionary times for exhaling and for inhaling. It was also identified that the air flow resistance for a certain volume is opposite to the time interval wherein this volume is displaced. The solution devised was to expand the process of exhaling in the inhaling interval, and expand the process of inhaling in the exhaling interval. Elastic walls were conceived in exhaling or inhaling chambers. Since the elastic rebound occurs in the phase opposite the concentration of elastic energy, the volume of exhaling is diluted in the inhaling time and the volume of inhaling is diluted in the exhaling time, reducing the air flow resistance and reducing respiratory fatigue. This solution may be efficiently applied to respirators or protective masks against contaminants with air filters. This will be explained in greater detail in the drawings.

These solutions can be used independently, but the combination of any of the elements produces a highly efficient effect in reducing respiratory fatigue, and one solution enables or potentializes the other.

The accompanying drawings schematically show the solutions proposed by this application.

FIG. 1 shows a side view of an air pump with external gearing.

FIG. 2 shows a side view of an air pump with internal gearing with sliding body.

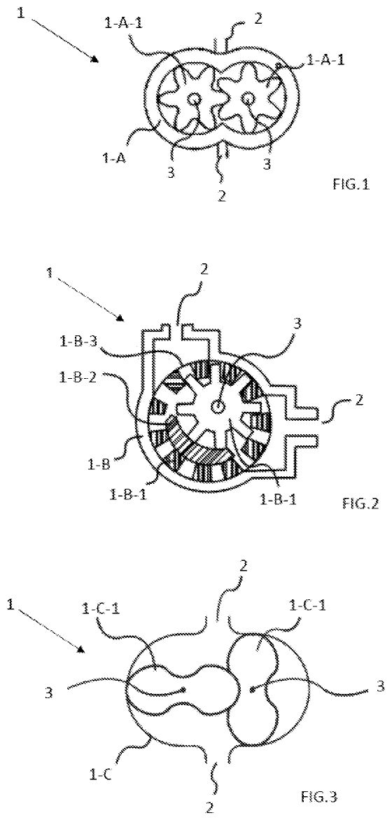

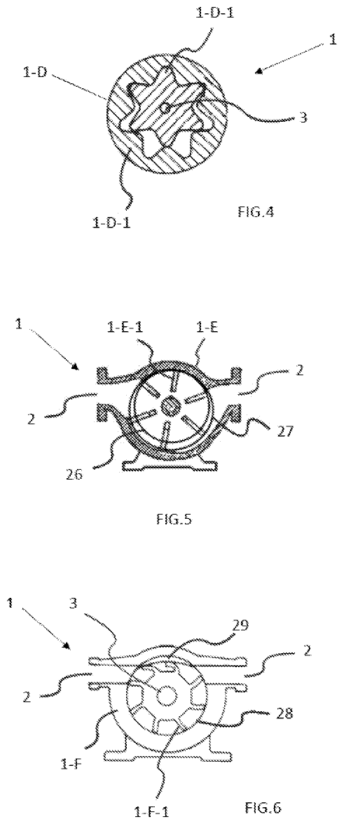

FIG. 3 shows a side view of a lobe air pump.

FIG. 4 shows a side view of another type of air pump with internal gearing with asymmetric center.

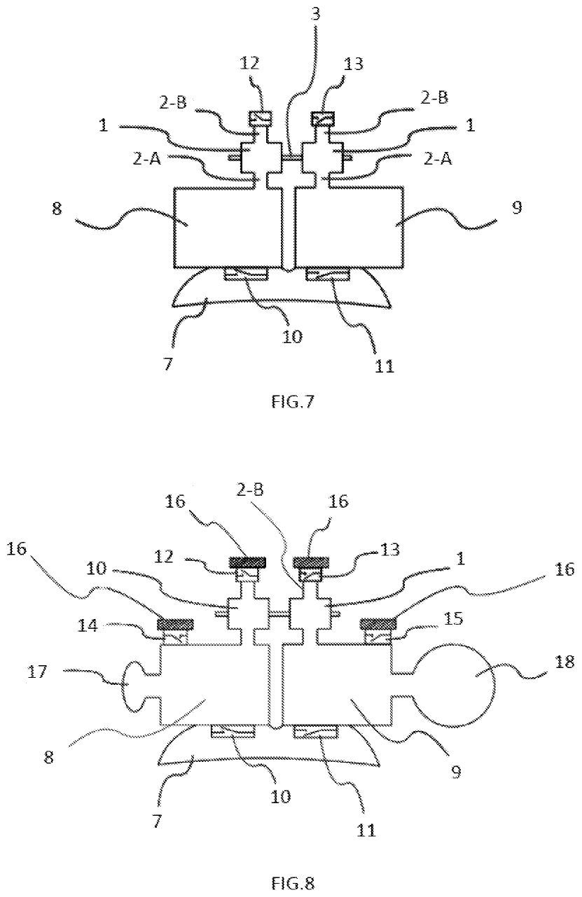

FIG. 5 shows a side view of a vane air pump.

FIG. 6 shows a side view of a flexible vane air pump.

FIG. 7 shows a side view of air distribution chambers coupled to air pressure transfer pumps.

FIG. 8 shows a side view of air distribution chambers coupled to air pressure transfer pumps, absorption walls and elastic rebound energy, escape valves and air filters.

FIG. 9 shows a side view of air distribution chambers with air filter inside the air chamber.

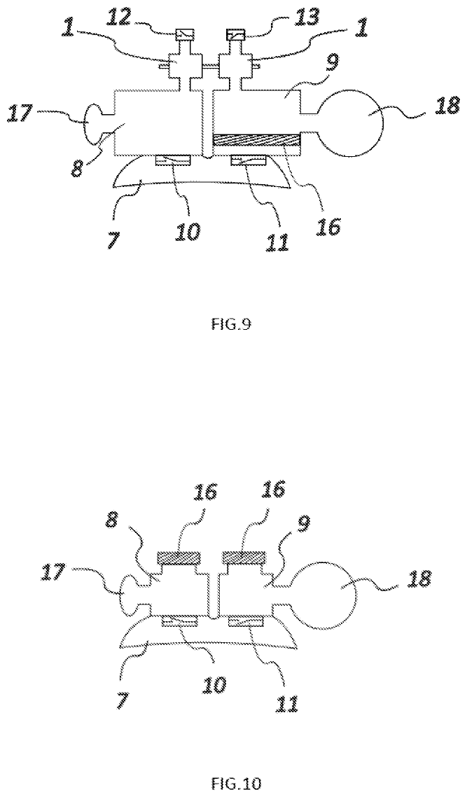

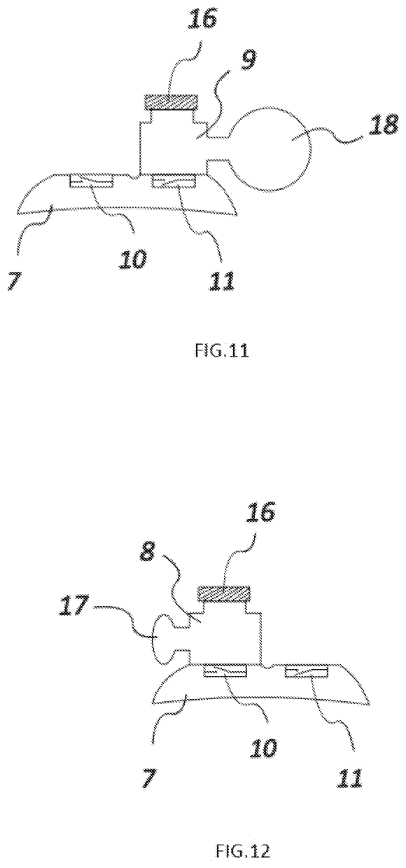

FIG. 10 shows a side view of air distribution chambers coupled to absorption walls of elastic energy applicable to the breathing phases of inhaling or exhaling. Their purpose is to reduce air flow resistance in masks or protective respirators against contaminants in the inhaling and exhaling phase.

FIG. 11 shows a side view of air distribution chambers coupled to absorption walls and elastic rebound energy applicable to the inhaling breathing phase. Their purpose is to reduce the air flow resistance in masks or protective respirators against contaminants in the inhaling phase.

FIG. 12 shows a side view of air distribution chambers coupled to absorption walls and elastic rebound energy applicable to the exhaling breathing phase. Their purpose is to reduce the air flow resistance in masks or protective respirators against contaminants in the exhaling phase.

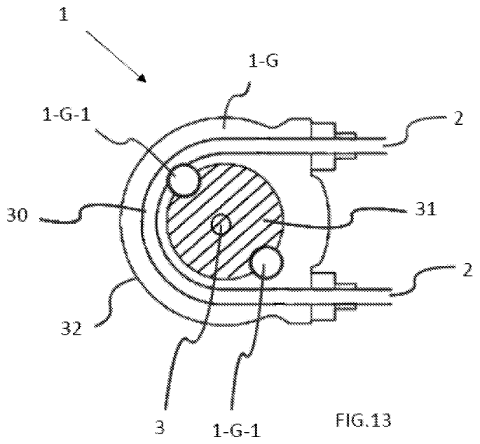

FIG. 13 shows a side view of a peristaltic air pump.

FIGS. 1, 2, 3, 4, 5, 6 and 13 are air pumps (1) of the rotary or circular type because they transform mechanical rotary energy around the axis (3) into production of difference in volume and air pressure, and vice-versa.

FIG. 1 illustrates a rotary or circular air pump (1) with external gearings (1-A-1), recognized as “air pump with external gearing” (1-A). The air flow enters by any one of the inlets (2) and leaves by another air outlet (2). The air passes through the sides of the two gearings and does not pass through the meeting of the cogs of the two gearings because at this point the passage of air is not possible. When the gearings turn, as the air is only conveyed over the sides of the gearings, the air passes in a single direction through the air inlets and outlets of the air pump with external gearing (1-A), producing pneumatic energy in the form of air flow. In the reverse way, upon injecting air through one of the air inlets (2) the gearings (1-A-1) turn, producing motive energy. It is an easy-to-build pump model, having high stability and low maintenance.

FIG. 2 illustrates a rotary or circular air pump (1) with internal gearings (1-B-1) whose cogs meet internally, also being recognized as an air pump with internal gearing with sliding body (1-B). The air flow enters through one of the inlets (2) and leaves by another air outlet (2). A sliding body (1-B-2) enables the cogs to separate and provides a smooth surface for conveying the volume of air along its surface. At the meeting points of the cogs of the internal gearings (1-B-1) there is no passage of air. Air is conveyed over the surface of the sliding body (1-B-2). There are air passage channels around the contours of the external rotor (1-B-3). So the air passes through the air passages and is conveyed from one point (2) to another point (2) of the air pump with internal gearing (1-B). Consequently motive energy can be transformed into pneumatic energy and vice versa. It is a highly efficient pump model, having low leakage and low attrition due to the fact of having less slant between the cogs of the two rotors (internal and external) at the meeting points of these cogs.

FIG. 3 illustrates a rotary or circular air pump (1) with lobules (1-C-1), also recognized as a lobe air pump (1-C). It has the same structure and function as the structure described in FIG. 1 with the difference of having lobes (1-C-1) in the place of cogs. It is an easy-to-build pump model, having high stability and low maintenance. It has less attrition than the pump of FIG. 1.

FIG. 4 illustrates a rotary or circular air pump (1) with gearings (1-D-1) that meet internally, also recognized as an air pump with internal gearing with asymmetric center (1-D). It has the same structure and function as the structure described in FIG. 2 with the difference of not requiring the sliding body (1-B-2). There is no passage of air at the meeting points of the cogs of the gearings (1-D-1). As the cogs separate, air can be conveyed, whereupon air flow occurs. So motive energy can be transformed into pneumatic energy and vice-versa. It is an easy-to-build pump model, having high stability and low maintenance.

FIG. 5 illustrates a rotary or circular air pump (1) of the vane type, also recognized as vane air pump (1-E). It consists of an external rotor (27) and an internal rotor (26). The vanes (1-E-1) are inserted into the internal rotor (26) and telescopically slide until they touch the internal face of the external rotor (27) promoting air sealing. The air circulates on the portions in which the two rotors (26 and 27) move apart and does not circulate where same (26 and 27) come together (or circulates in lesser volume). The air is conveyed with the rotation of the system between the inlet (2) and outlet (2) channels in pockets of air produced by the distancing of the two rotors (26 and 27). There are pneumatic communications between the air channels (2) and the inside of the external rotor (27). So motive energy can be transformed into pneumatic energy and vice versa. It is a highly efficient pump model, having low attrition.

FIG. 6 illustrates a rotary or circular air pump (1) of the flexible vane type, also recognized as flexible vane air pump (1-F). An axis rotor (3) having flexible vanes (1-F-1) turns internally in a cylindrical cavity (28), this cavity (28) having a protruding body (29) which folds the vanes (1-F-1) when they pass by its position. Therefore, in this region (29) the air is impeded by a reduction of air volume at the site, and the air is obliged to head to one of the side air outlets (2) and captures air on the opposite side on the inlets (2) by increasing the volume between the vanes (1-F-1) by unfolding the vanes (1-F-1). So motive energy can be transformed into pneumatic energy and vice versa. This is one of the preferred forms to be used in the solutions to be presented in the coming drawings. This choice is due to the ease of construction of this type of pump, ease of cleaning and decontamination, use of a fewer number of rotors relative to the solutions of the preceding drawings obtaining greater lightness.

FIG. 7 illustrates a face mask (7) which couples to the mouth, nose or face and connects to the chamber for exhaling (8) having a valve (10) that enables the one-way passage of air towards the face mask (7) to the inside of the chamber (8). It also has a chamber for inhaling (9) having a valve (11) that enables the one-way passage of air leaving the inside of the chamber (9) to the face mask (7). Rotary air pumps (1) of any type, such as described in FIGS. 1 to 6, are inserted having air communication channels (2-A) with the chambers for exhaling (8) or inhaling (9). At the outermost points, outlets (2-B) are illustrated, which release into or absorb air from the atmosphere. A rotary axis (3) transfers the rotation of the axes (3) of each air pump (1), transferring the mechanical energy between the air pumps (1) such that the air of the chambers (8 and 9) can be moved without mixing the air between them.

Further as illustrated in FIG. 7, in exhaling the air passes from the face mask (7) to the inside of the air distributor chamber (8) which releases the air into the environment, passing through the air pump (1), making its internal axis (3) turn. The mechanical energy of rotation passes to the air pump (1) connected to the inhaling chamber (9) that forces the air into this chamber (9) creating positive pressure. The flow valve (13) bars the exit of air from the inhaling chamber (9) maintaining positive pressure. This positive pressure assists the process of inhaling. This configuration enables positive pressure both in the exhaling phase and in the inhaling phase, being one of the preferred forms of using the solutions of this application.

Further as illustrated in FIG. 7, another form of using this solution is in the production of negative pressure in the exhaling phase. Put otherwise, starting from inhaling, negative pressure is created in the inhaling chamber (9), the air pump (1) transfers mechanical energy to another pump (1) by means of a common axis (3), generating negative pressure in the exhaling chamber (8) which may facilitate some situations of exhaling (useful in pathologies with difficulties in exhaling such as in emphysemas or in rib cage alterations). The valve (12) prevents the loss of negative pressure in the chamber (8).

Further as illustrated in FIG. 7, many variations may be applied by changing the direction of the rotation axis (3) or modifying the air communication with the air inlets and outlets (2) and air pumps (1). The rotation axes (3) may be substituted by any mechanical communication or even by mechanical energy transferring magnetic materials. Magnetic transmission is useful to prevent the exchange of air between chambers and increasing sealing and safety. Mechanical locks that only allow rotation in one direction may be inserted in the rotation axes (3) to enable maintenance of the differences in air pressure. An air pump is understood to be any device that transforms air flow into motive force and vice versa, and may substitute the rotary pumps (1) in this application.

Air pumps may be of any type (rotary or non-rotary) including the ones described in FIG. 1, 2, 3, 4, 5 or 6, also including centrifugal, axial or peristaltic types (FIG. 14). Preferably but not exclusionary, in this application, it was established that the most recommended air pumps are the rotary ones because they are lighter and adapt well to diverse structures. The option of promoting rotary pumps (1) was envisaged because these rotary pumps occupy little space and enable regulation adapted to low volumes of air as opposed to other air pumps such as piston-based ones. Furthermore, the passage of mechanical energy by means of rotary axis (3) is quite simplified and functional, necessary for the lightness and durability of the solution proposed. Another preferred solution is the peristaltic air pump (FIG. 13) with two internal hoses and those having flexible vanes (FIG. 6) being an easy-to-build solution and using little material, being very light.

As illustrated in FIG. 8, the same components described in FIG. 7 appear, but with the addition of pressure equalization valves (14 and 15) which are opened when the pressure differences exceed a certain value. One of the drawbacks that the action of the air pumps (1) may produce is to create a negative environment (when undesirable) in the air chambers (8 and 9) which may cause collapse of the alveoli or of the respiratory tracts or difficulty getting air in on inhalation, an undesirable condition in some pathologies such as obstructive sleep apnea. In one of the preferred arrangements, these valves (14 and 15) release air flow in a one-way direction from the outside environment to the internal environment when there is a reduction in pressure inside the chambers (8 or 9). They can be used to prevent negative air pressure in the air chambers (8 or 9) or be safety exhausts for air outlet (changing the direction of the valve) when there is excess pressure in the chambers (8 or 9) the admission of other types of values being established such as safety or relief valves. One interesting valve is that which opens when the pressure is very low and when it is very high.

As further illustrated in FIG. 8, air expansion or retraction chambers (17 and 18) are added, containing elastic walls connected to the exhaling or inhaling chambers (8 or 9). The elastic-wall chambers (17 and 18) serve to store the air pressure in elastic energy, decreasing the respiratory effort. The elastic chamber (17) was devised so that part of the volume stored in the exhaling phase does not have to be fully transferred in the exhaling, since the chamber (17) enables elastic rebound such that the air stored is transferred in the other phase, that of inhaling, when the pressure is reduced in the inhaling chamber (9) during inhaling. Additionally, air filters (16) increase the air resistance, and these chambers (17 and 18) minimize this additional respiratory effort during exhaling or inhaling. The elastic chamber (18) serves to expand, capturing air coming from the air pumps (1) during exhaling. During inhaling, this accumulated air in the form of positive pressure in the chambers (9 and 18) facilitates inhaling. Further during inhaling, once the positive pressure is exhausted in the inhaling chamber (9), soon afterwards the chamber (18) can elastically retract by negative pressure minimizing the air resistance produced by the air filter (16) during inhaling and returning this energy in another phase (in exhaling) as the elastic chamber (18) expands, returning to the initial volume in the exhaling phase and begins to capture air from the environment by the air inlet route (2-B). Accordingly, the energy that is elastically concentrated in the inhaling passes onto the exhaling phase and vice versa, decreasing the respiratory effort. The walls of the chambers (8 or 9) themselves were conceived to have elastic walls producing the effect of the elastic chambers (17 or 18).

Further as illustrated in FIG. 8, air filters (16) are also added to the air inlets or outlets. They serve to reduce the intake or output of contaminants from the system, avoiding contamination of the respirator user or contaminations to other people by biological agents coming from the user him/herself. In situations of contaminated external environments, the main objective of the device can be to protect the user against contaminants. Put otherwise, the main purpose of the preceding solutions was to reduce air flow resistance to assure the feasibility of use of more efficient and more comfortable breathing filters, preventing respiratory fatigue.

As further illustrated in FIG. 7 or 8, some valves and filters may be withdrawn according to the function.

As illustrated in FIG. 9, the same components described in FIG. 8 appear, but with the air filter (16) inserted inside the air chambers (8 or 9) which facilitates accommodation of the air filters (16).

FIG. 10 illustrates a face protection mask or respirator against physical, chemical or biological contaminants, to prevent contamination of the user or other people, based on a barrier air filter (16). A face mask (7) was devised to be coupled to the mouth, nose or face having a chamber for exhaling (8) with a valve (10) that only allows the passage of air from the face mask (7) to the inside of the chamber (8), air filter (16) and elastic expansion chamber (17) having elastic walls. It also has a chamber for inhaling (9) having valve (11) that only allows the passage of air leaving the inside of the chamber (9) towards the face mask (7), air filter (16) and elastic retraction chamber (18) having elastic walls.

As further illustrated in FIG. 10, upon exhaling air passes from the face mask (7) to the inside of the exhaling air chamber (8), part of the air leaving by the filter (16). Part of the air goes to the expansion chamber (17), relieving the pressure of the distributor chamber (8). The volume of air accumulated in the expansion chamber (17) is stored to be released in the other phase of the breathing which is the inhaling. Accordingly, the effort to exhale the air is reduced.

Further as illustrated in FIG. 10, upon inhaling, air enters the face mask (7), leaving the inside of the distributor chamber (9), part of the air entering by the filter (16). Part of the air comes from the elastic retraction chamber (18) the retraction of which relieves the negative pressure of the distributor chamber (9). During inhaling, the elastic retraction chamber (18) contracts, reducing its volume. After inhaling, in the exhaling phase, this chamber (18) returns to the initial volume by expansion through elastic rebound and receives air through the air filter (16). Accordingly, the effort to inhale the air is reduced.

As illustrated in FIG. 11, the arrangement is to prevent the user from being contaminated, having the same structure as that of FIG. 10, but containing only the component for the inhaling phase without the component of the exhaling phase. In the exhaling phase, the air exits directly into the environment through the valve (10). The air filtering is intended solely for the inhaling phase, the device idealized creates less air resistance and less air flow speed which generates more breathing comfort and lower penetration force by contaminants through the filter (16), increasing user safety.

As illustrated in FIG. 12, the structure is the same as that of FIG. 10, but containing only the component for the exhaling phase without the component of the inhaling phase. In the inhaling phase, the air enters directly from the environment through the valve (11). Ideal arrangement for whoever is already contaminated by some micro-organism, such as, for example, Covid-19, and cannot contaminate others. The air filtering is intended solely for the exhaling phase, the device idealized creates less air resistance and less air flow speed which generates breathing comfort and less air turbulence with less dispersion of contaminants.

Further as illustrated in FIG. 10, 11 or 12, both the expansion chamber and the elastic retraction chamber (17 or 18) have elastic walls which may be any shape, such as in the form of bag, bellows, concertinas or even in the form of pistons. The chambers (8 or 9) themselves may have elastic walls. The material can be any type, such as elastic, plastic, or a combination thereof. Many variations are acceptable, the importance being that they have the elastic property of deforming and rebounding to the prior volume in the various phases of inhaling and exhaling.

FIG. 13 illustrates an air pump of the peristaltic type (1-G) in which a flexible air hose (30) is arranged inside a cylinder bed body (32) having a rotor (31) with cog substitutes in the form of rollers (1-G-1) which upon turning around its axis (3) presses this hose (30) on the walls of the body (32) producing air flow in the direction of the rotation of the rotor (31) having an air inlet and outlet port (2). The system also allows the injection of air into the hose (30) to cause movement of the rotor (31) and rollers (1-G-1). A second hose (30) can be introduced into the bed of the inner body of the body (32) in the same space as a first hose (30) such that one hose (30) receives air and the other provides air.

Accordingly, this type of pump makes it possible to have in a same rotary structure (31) a system of transforming pneumatic energy into mechanical energy, and vice versa, economizing on material and providing lightness and facility of construction, being one of the lightest structures and easy to maintain among those presented. The system of hoses (30) provides easy management of adjustment gaps, prevents air leakage, allows easy disinfection and change of parts. The system connects to the structures of the FIG. 7 or 8 through the airways (2). The air pump type is a preferred use for this application. In the arrangement using two hoses (30) on the same cylinder bed (32) or in the arrangement using different cylinder beds (32), for both arrangements, one of the hoses (30) connects to the air outlet (2-A) of the exhaling chamber (8) illustrated in FIG. 7 and the other different hose (30) connects to the air inlet (2-A) of the inhaling chamber (9). The other ends of these two different hoses (30) connect with the outside environment by the air outlet and inlet ducts (2-B).

According to any one of the figures, the constitution of the masks and respirators get mixed up. The model presented by this application resembles more face respirators, but some of these may be quite moldable on the contours of the face that its concept gets mixed up with respiratory masks, the objective of this application being adapted for both items of equipment. The terms respirator and mask can be used as synonyms in this application. Pumps of different types may be combined.

The components of this application can be produced in a single body or in separate and interchangeable components.

1. A device for transferring energy between exhaling and inhaling phases comprising: a rotary air pumps (1), exhaling chamber (8) and inhaling chamber (9) and one-way air valves (10) and (11) directly connected to a face mask (7), wherein the one-way air valves (10) and (11), connected independently to the air distribution chamber for exhaling (8) and to the air distribution chamber for inhaling (9), respectively; the exhaling (8) and inhaling (9) chambers are each connected through an air communication channel (2-A) to the rotary air pumps (1) containing a single rotary axis (3), connected simultaneously to the two pumps (1), which transfers the energy between the air pumps (1) and wherein said air pumps (1) are connected to the outside environment by way of an air outlet and inlet ducts (2-B).

2. The device according to claim 1 comprising a valve (12 or 13) in the air path (2-A or 2-B) that passes through the rotary pumps (1).

3. The device according to claim 1 wherein the air pump (1) is of the external gearing type (1-A) with external gearing (1-A-1).

4. The device according to claim 1 wherein the air pump (1) is of the internal gearing slide type (1-B) comprised with internal gearing (1-B-1) with sliding body (1-B-2) and external rotor (1-B-3).

5. The device according to claim 1 wherein the air pump (1) is of the lobular type (1-C) comprising lobes (1-C-1).

6. The device according to claim 1 wherein the air pump (1) is of the internal gearing type with asymmetric center (1-D) comprising gearings (1-D-1).

7. The device according to claim 1 wherein the air pump (1) is of the vane air pump type (1-E) comprising vanes (1-E-1).

8. The device according to claim 1 wherein the air pump (1) is of the flexible vane air pump type (1-F) comprising flexible vanes (1-F-1).

9. The device according to claim 1 wherein the air pump (1) is of the peristaltic type (1-G) comprising rollers (1-G-1), two or more hoses (30) inside the rotary cylinder (32).

10. The device according to claim 1 comprising an air distribution chamber (8 or 9) having at least one of the following items, expansion or elastic retraction chambers (17 or 18).

11. The device according to claim 1 comprising an air pressure escape valve (14 or 15) located between the air distribution chamber (8 or 9) and the atmospheric air.

12. The device according to claim 11 comprising an air filter (16) in at least one of the locations cited ahead, in the air inlet or outlet of the air distribution chamber (8 or 9), inside the air distribution chamber (8 or 9), in the air inlet or outlet of the air pump (1) or in the air inlet or outlet of the escape valve (14 or 15).
